(19) United States
(12) Patent Application Publication
(10) Pub. No.: US 2015/0352346 A1
(43) Pub. Date: Dec. 10, 2015

(54) Tattoo Needle Drive System

(71) Applicant: Raymond D. Webb, Miami, AZ (US)

(72) Inventor: Raymond D. Webb, Miami, AZ (US)

(21) Appl. No.: 14/729,926

(22) Filed: Jun. 3, 2015

Related U.S. Application Data
(60) Provisional application No. 62/007,462, filed on Jun. 4, 2014.

(51) Int. Cl. A61M 37/00

(52) U.S. Cl. CPC A61M 37/0076

(57) ABSTRACT

A tattoo needle drive system for providing adjustment of the firmness of a needle strike into the skin. The tattoo needle drive system generally includes a drive unit having a frame and a motor and a drive member movably connected to the drive unit. The motor is mechanically connected to the drive member and is adapted to reciprocate in a back-and-forth linear motion. A connecting member is removably connected between the tattoo needle and the drive member to transfer the back-and-forth linear motion from the drive member to the tattoo needle. The connecting member is constructed of a resilient material such as plastic or rubber to provide a desired firmness to the needle strike into the skin.

### CROSS REFERENCE TO RELATED APPLICATIONS

I hereby claim benefit under Title 35, United States Code, Section 119(e) of U.S. provisional patent application Ser. No. 62/007,462 filed Jun. 4, 2014. The 62/007,462 application is currently pending. The 62/007,462 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a tattoo machine and more specifically it relates to a tattoo needle drive system for providing adjustment of the firmness of a needle strike into the skin.

2. Description of the Related Art

Any discussion of the related art throughout the specification should in no way be considered as an admission that such related art is widely known or forms part of common general knowledge in the field.

Conventional tattoo machines are hand held devices that move needles in a reciprocating manner to deposit ink into the dermis layer of skin with each insertion. There are two main types of tattoo machines: coil tattoo machines and rotary tattoo machines. There are other types of tattoo machines such as pneumatic tattoo machines powered by an air compressor, shader tattoo machines and liner tattoo machines.

Conventional coil tattoo machines utilize a pair of electromagnetic coils to trigger a draw and release of the tattoo machines armature bar creating a hammer-like effect where the armature bar taps the attached tattoo needle into the skin. U.S. Pat. No. 8,171,825 to James Adams illustrates a Magnetic Coil Tattooing Machine and other conventional coil tattoo machines.

Conventional rotary tattoo machines utilize a regular electric motor or pneumatic motor that rotates a drive shaft to move a mechanism in a reciprocating manner. There are four main types of conventional rotary tattoo machines: direct drive rotary tattoo machines, armature bar rotary tattoo machines, linear action rotary tattoo machines and swashdrive rotary tattoo machines. U.S. Pat. No. 8,733,211 to Michael Snijders, U.S. Pat. No. 7,207,242 to Ronald Daigle and U.S. Pat. No. 8,522,647 to Alan Dixon illustrate examples of conventional rotary tattoo machines.

One of the problems with conventional tattoo machines is the limited ability for the tattoo artist to optimize the firmness of the needle strike into the skin. Tattoo artists often times have a different style and preference regarding the firmness of how a needle strikes the skin of the customer.

Because of the inherent problems with the related art, there is a need for a new and improved tattoo needle drive system for providing adjustment of the firmness of a needle strike into the skin.

### BRIEF SUMMARY OF THE INVENTION

The invention generally relates to a tattoo machine which includes a drive unit having a frame and a motor and a drive member movably connected to the drive unit. The motor is mechanically connected to the drive member and is adapted to reciprocate in a back-and-forth linear motion. A connecting member is removably connected between the tattoo needle and the drive member to transfer the back-and-forth linear motion from the drive member to the tattoo needle. The connecting member is constructed of a resilient material such as plastic or rubber to provide a desired firmness to the needle strike into the skin.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a lower perspective view of the present invention.

FIG. 2 is an exploded lower perspective view of the present invention.

FIG. 3 is an upper perspective view of the present invention.

FIG. 4 is a side view of the present invention.

FIG. 5 is a front view of the present invention.

FIG. 6 is a cross sectional view taken along line 6-6 of FIG. 5.

FIG. 7a is a cross sectional view of a first alternative embodiment of the present invention.

FIG. 7b is a cross sectional view of a second alternative embodiment of the present invention.

FIG. 8 is a lower perspective view of a third alternative embodiment of the present invention.

FIG. 9 is an exploded lower perspective view of the third alternative embodiment of the present invention.

FIG. 10 is an upper perspective view of the third alternative embodiment.

FIG. 11 is a side view of the third alternative embodiment.

FIG. 12 is a front view of the third alternative embodiment.

### DETAILED DESCRIPTION OF THE INVENTION

### A. Overview

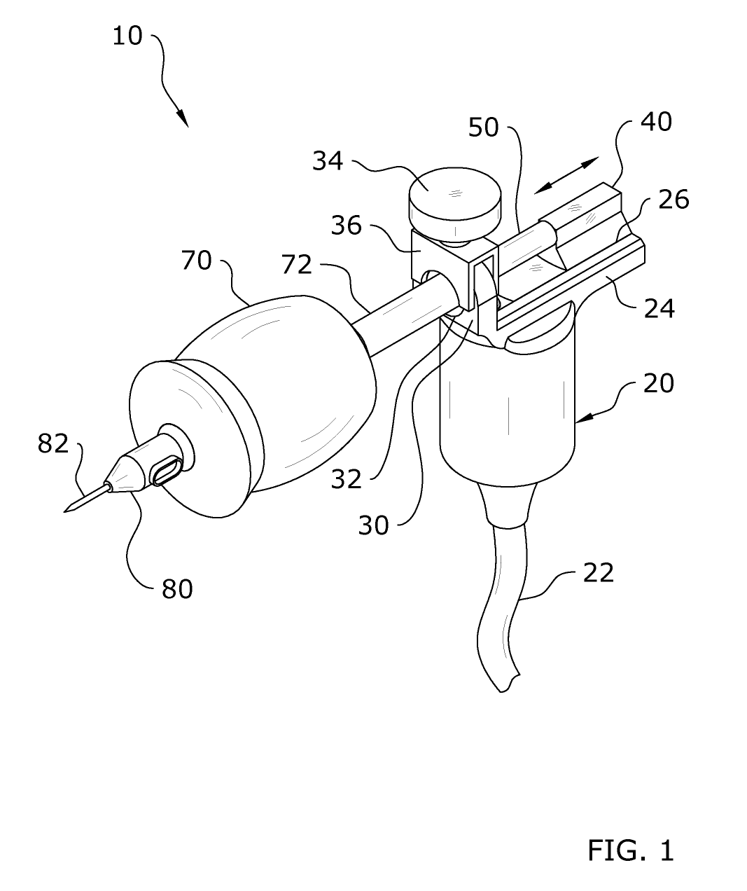

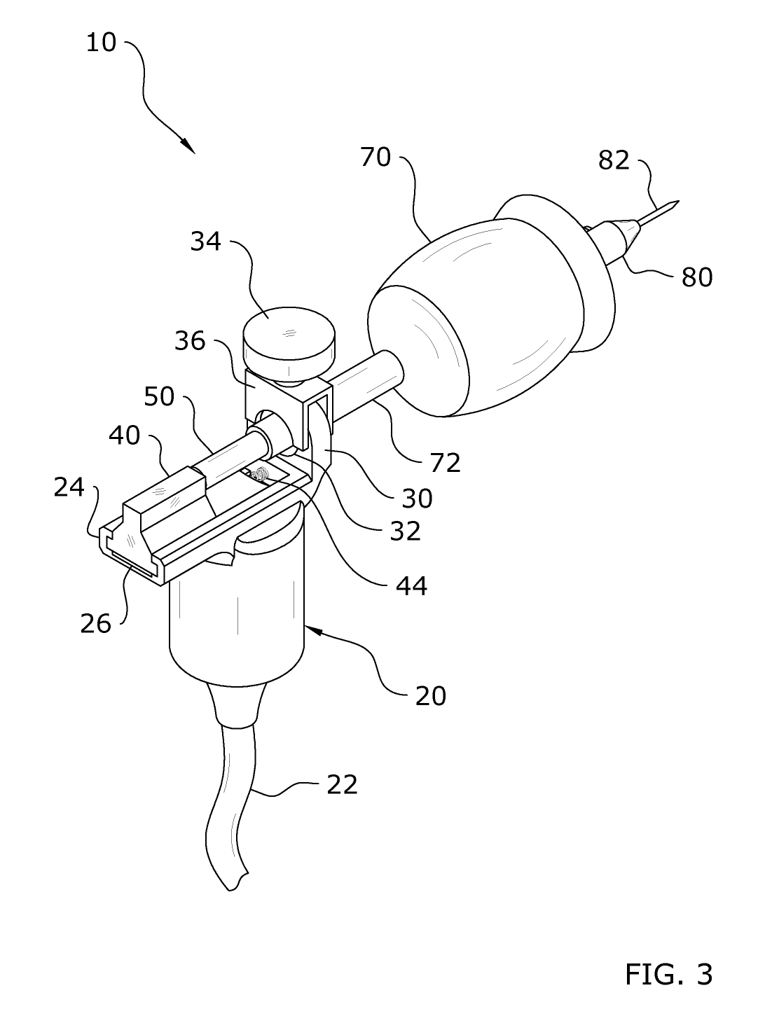

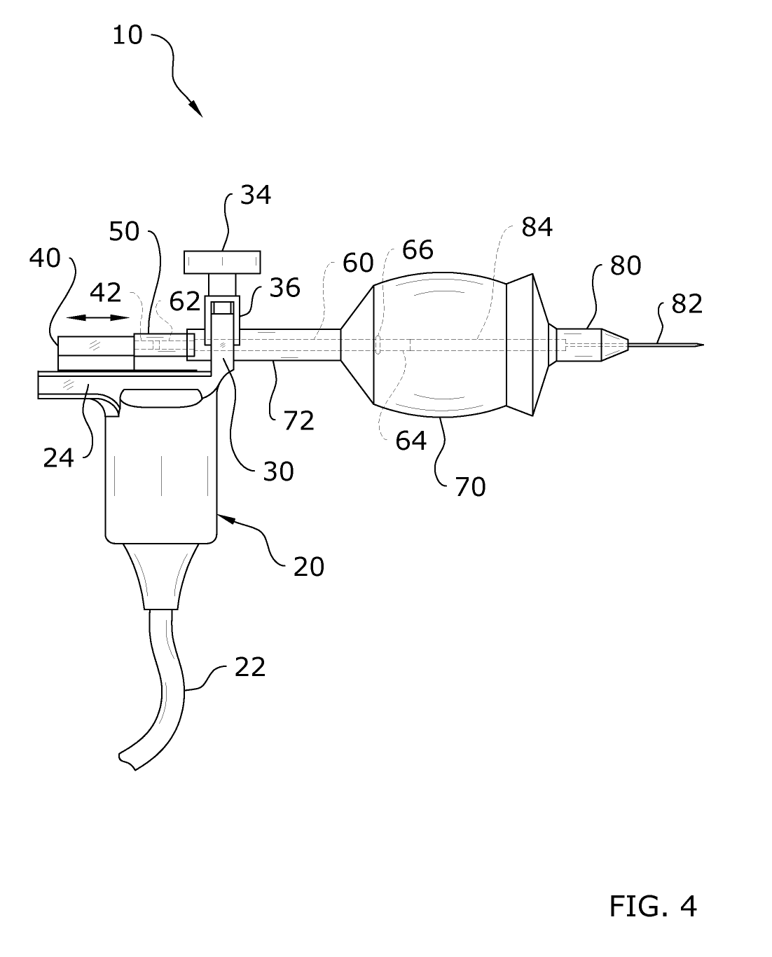

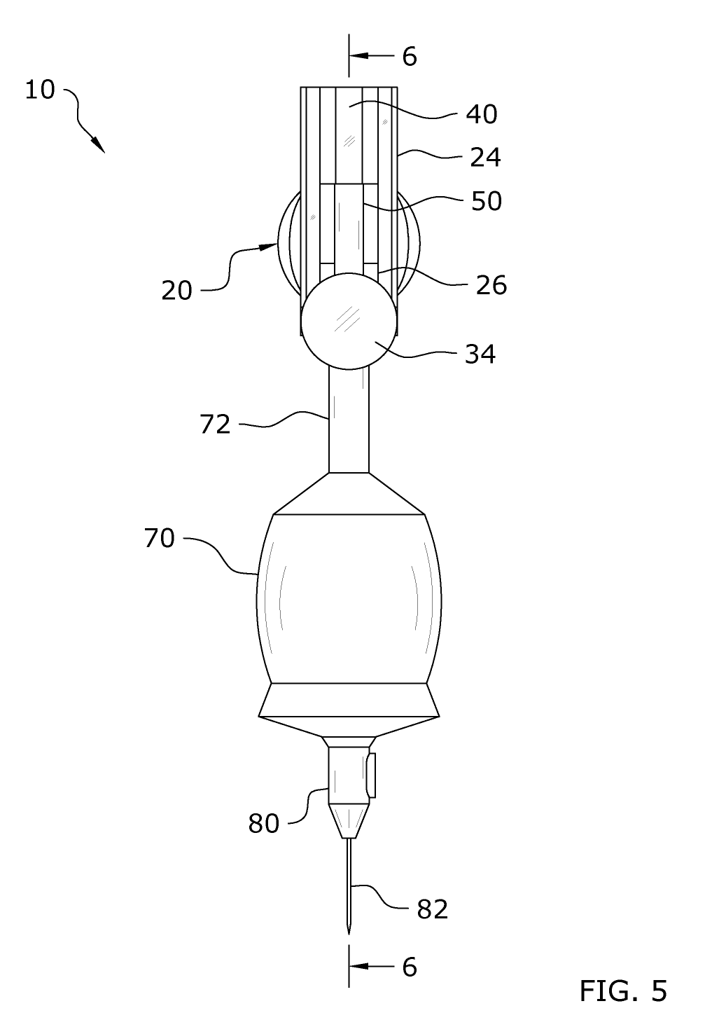

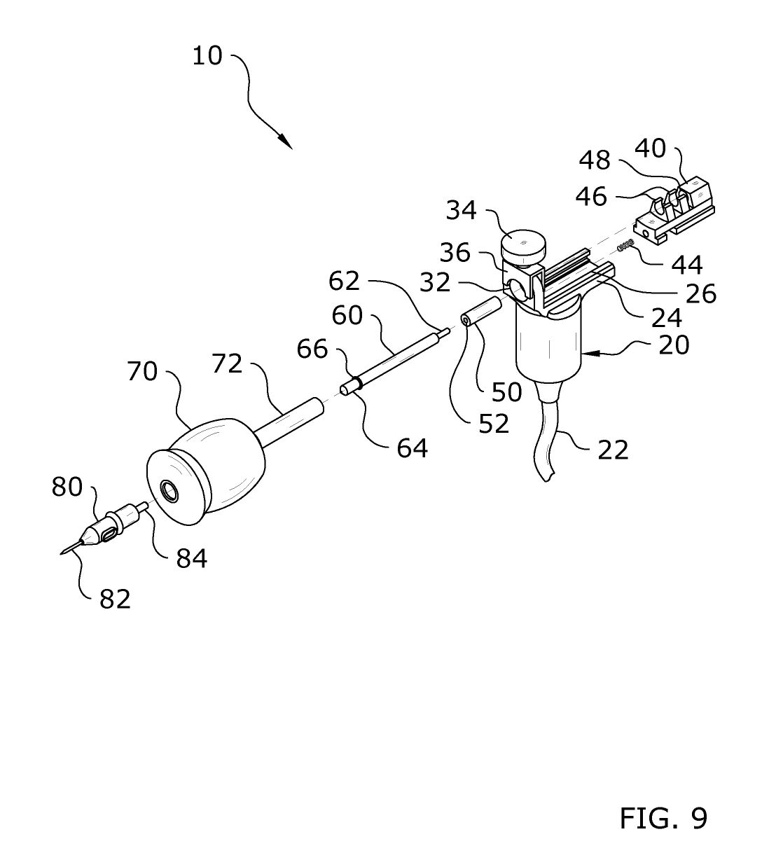

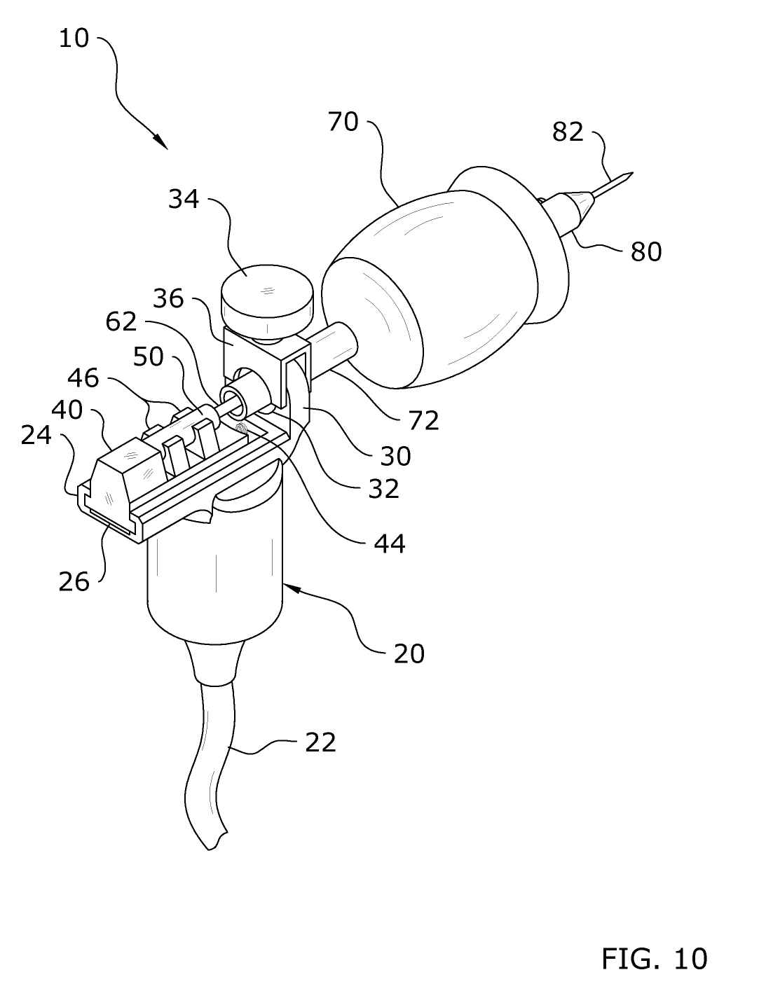

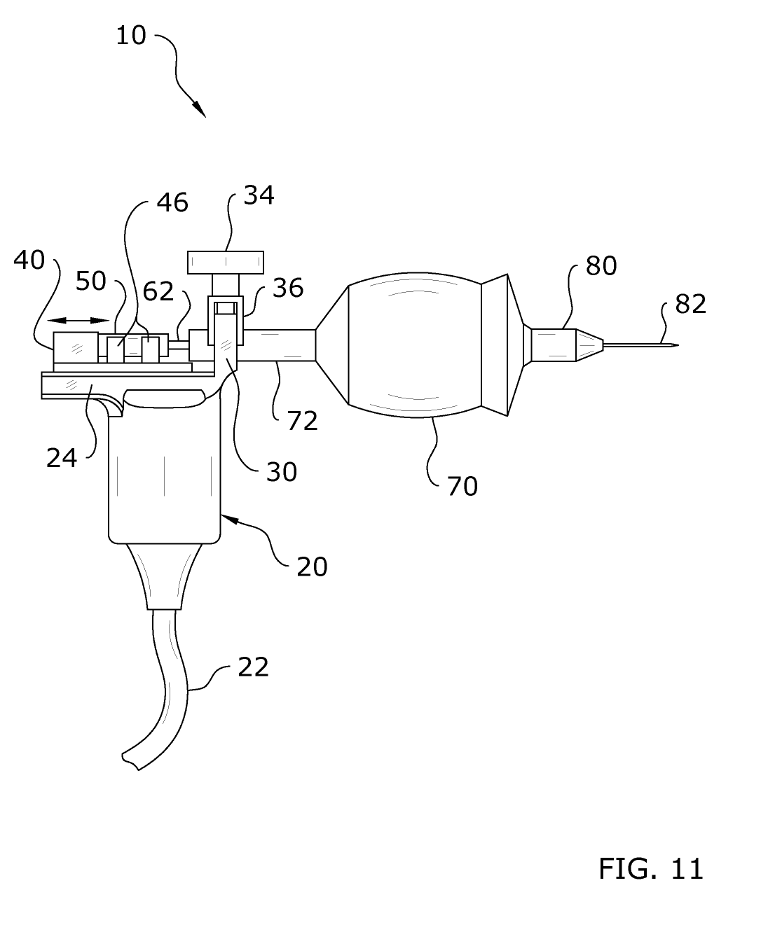

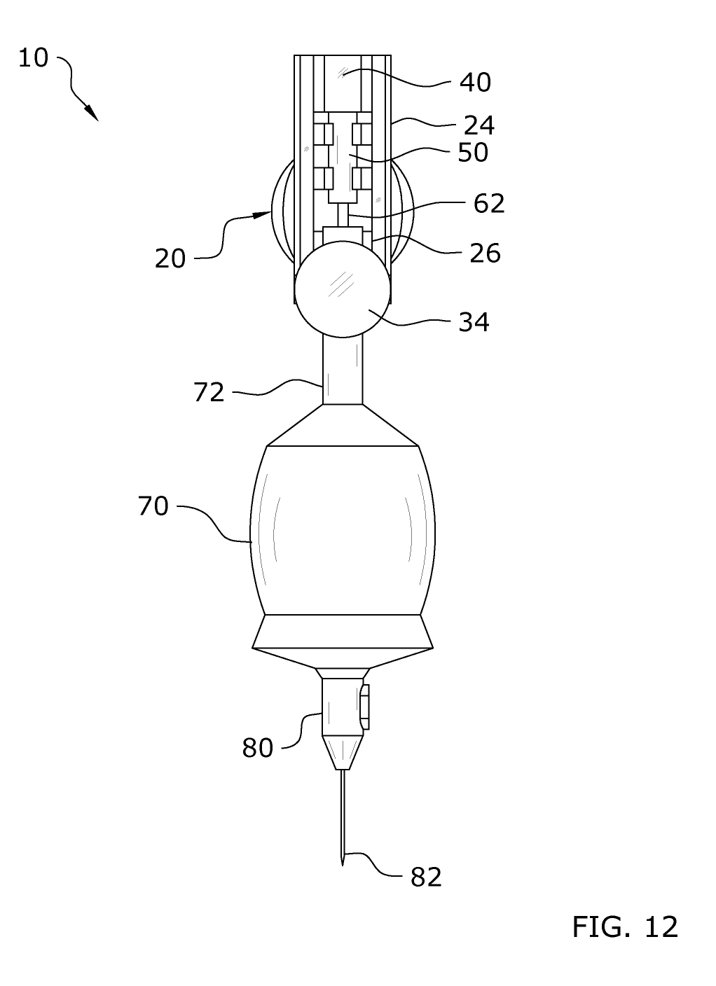

Turning now descriptively to the drawings, in which similar reference characters denote similar elements throughout the several views, FIGS. 1 through 12 illustrate a tattoo needle drive system 10, which comprises a drive unit 20 having a frame 24 and a motor 21 and a drive member 40 movably connected to the drive unit 20. The motor 21 is mechanically connected to the drive member 40 and is adapted to reciprocate in a back-and-forth linear motion. A connecting member 50 is removably connected between the tattoo needle 82 and the drive member 40 to transfer the back-and-forth linear motion from the drive member 40 to the tattoo needle 82. The connecting member 50 is constructed of a resilient material such as plastic or rubber to provide a desired firmness to the needle strike into the skin. The present invention is preferably a hand held device that moves a conventional tattoo needle 82 in a reciprocating manner to deposit ink into the dermis layer of skin with each insertion.

### B. Drive Unit

The drive unit 20 provides the reciprocating motion to the tattoo needle 82 with the connecting member 50 positioned between the drive unit 20 and the tattoo needle 82 to provide a desired firmness of needle strike into the skin of the customer. The drive unit 20 moves a drive member 40 in a reciprocating back-and-forth motion thereby driving the tattoo needle 82 into the skin of the customer of the tattoo artist. The connecting member 50 positioned between the drive member 40 and the tattoo needle 82 is comprised of a resilient material that absorbs a portion of the impact force from the drive member 40 to create the desires level of firmness of needle strike into the skin of the customer.

The drive unit 20 when combined with the tattoo needle 82 and other components necessary to form a tattoo machine, may be comprised of a coil tattoo machine, a rotary tattoo machine, a pneumatic tattoo machine, a shader tattoo machine or a liner tattoo machine. A power cord 22 is electrically connected to an electrical receptacle to provide electrical power to the electric motor 21. It is preferable that the drive unit 20 forms a rotary tattoo machine such as, but not limited to, a direct drive rotary tattoo machine, an armature bar rotary tattoo machine, a linear action rotary tattoo machine or a swashdrive rotary tattoo machine. It is further preferable that the drive unit 20 is used to form a linear action rotary tattoo machine as illustrated in FIGS. 1 through 12 of the drawings. The following patents are incorporated by reference herein: U.S. Pat. No. 8,171,825 to James Adams, U.S. Pat. No. 8,733,211 to Michael Snijders, U.S. Pat. No. 7,207,242 to Ronald Daigle and U.S. Pat. No. 8,522,647 to Alan Dixon.

The drive unit 20 is comprised of a frame 24 and a motor 21 attached to the frame 24 as illustrated in FIGS. 1 through 12 of the drawings. FIGS. 1 through 4 and 6 through 11 illustrate the frame 24 of the drive unit 20 having a tubular main body with a lower opening that removably receives the motor 21. The frame 24 may be constructed of various types of rigid non-resilient materials such as metal or hard plastic.

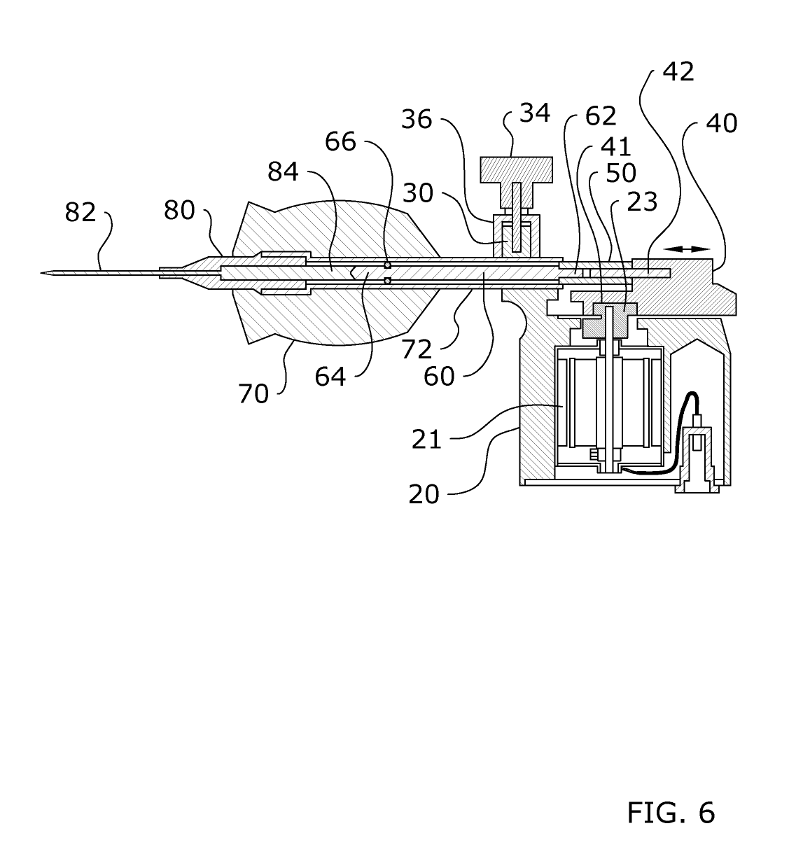

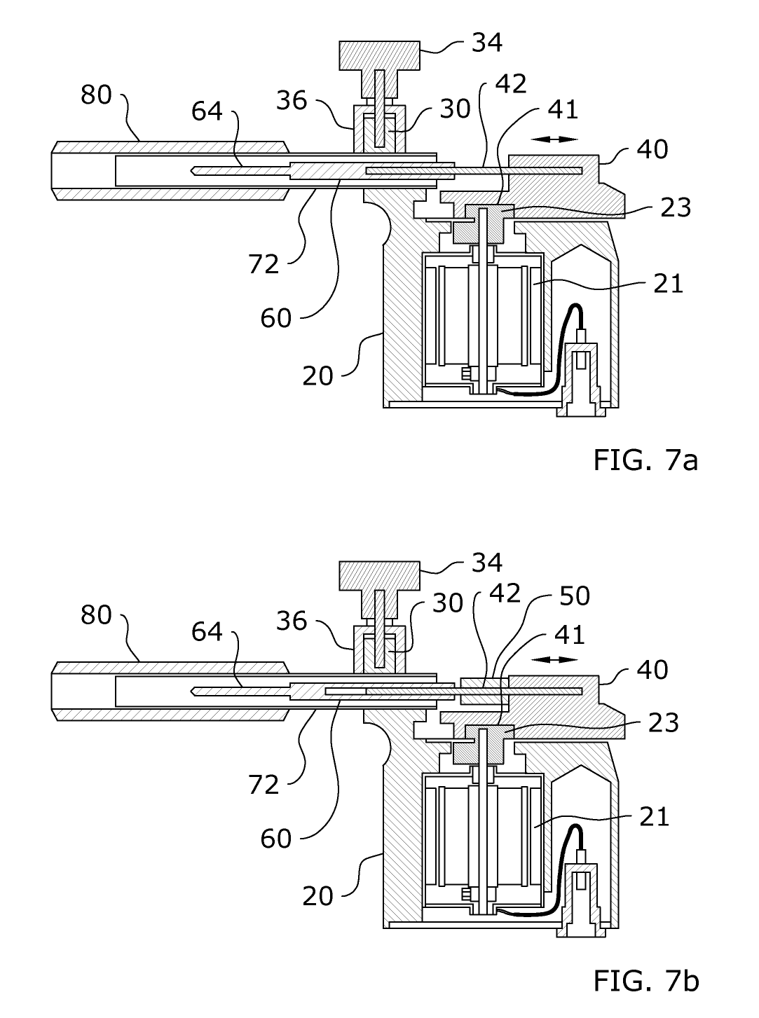

The motor 21 is preferably a rotary motor 21 having a rotating drive shaft. The rotary motor 21 may be any type of rotating motor 21 such as, but not limited to, an electric motor 21 or a pneumatic motor 21. A cam 23 is attached to the drive shaft so that the cam 23 forces the drive member 40 back-and-forth in a reciprocating motion. The cam 23 is movably received within a cutout 41 within the drive member 40 as illustrated in FIGS. 6 through 7b of the drawings. The cutout 41 may have various shapes to provide various types of impacts and hammering effects on the tattoo needle 82. The cam 23 may be constructed of various types of cam structures, however, the cam 23 is preferably comprised of a cam wheel having a rotating ring on a bearing to assist in reducing friction between the cam 23 and the drive member 40.

A support tube 72 is attached to the drive. The support tube 72 is preferably constructed of a rigid material such as hard plastic or metal. The support tube 72 may include a handle 70 with rubberized gripping or other type of gripping structure for the tattoo artist to hold during the tattooing procedure.

The support tube 72 is preferably attached in a removable and/or adjustable manner to the drive unit 20 which is well known in the art of tattoo machines. FIGS. 1 through 12 illustrate the drive unit 20 having a mounting portion 30 extending from the frame 24 with a mounting aperture 32 that the support tube 72 extends through in an adjustable manner. A mounting fastener 34 is threadably connected to a threaded aperture within the mounting portion 30 to apply force to a mounting bracket 36 that is slidably connected to the mounting portion 30. The mounting bracket 36 is forced towards engagement of the support tube 72 to prevent movement of the support tube 72 within the mounting portion 30 after the mounting fastener 34 is tightened. The support tube 72 includes a proximal opening and a distal opening. The distal opening within the support tube 72 is formed and adapted to removably receive a conventional needle cartridge 80.

### C. Drive Member

A drive member 40 is slidably positioned in a channel 26 in the frame 24 of the drive unit 20 as illustrated in FIGS. 1 through 4 of the drawings. The channel 26 preferably has a T-shaped cross sectional structure with a portion of the drive member 40 having a corresponding T-shaped cross sectional structure that slidably moves within the channel 26.

The motor 21 is mechanically connected to the drive member 40 and the drive member 40 is adapted to reciprocate in a back-and-forth linear motion along a first axis that is preferably parallel to or aligned with a longitudinal axis of the tattoo needle 82. The drive member 40 is preferably constructed of a rigid non-resilient material such as metal or hard plastic. A lubricant is preferably positioned between the drive member 40 and the channel 26 to reduce friction. The cutout 41 in the inner side of the drive member 40 receives the cam 23 to transfer the rotating force of the cam 23 to a linear reciprocating motion for the drive member 40. Various other structures may be used to transfer the rotating force of the motor 21 to a linear reciprocating motion. A compression spring 44 is preferably positioned between the drive member 40 and an end of the channel 26 to apply a force to the drive member 40 that extends away from the striking movement of the drive member 40 when extending the tattoo needle 82 from the needle cartridge 80.

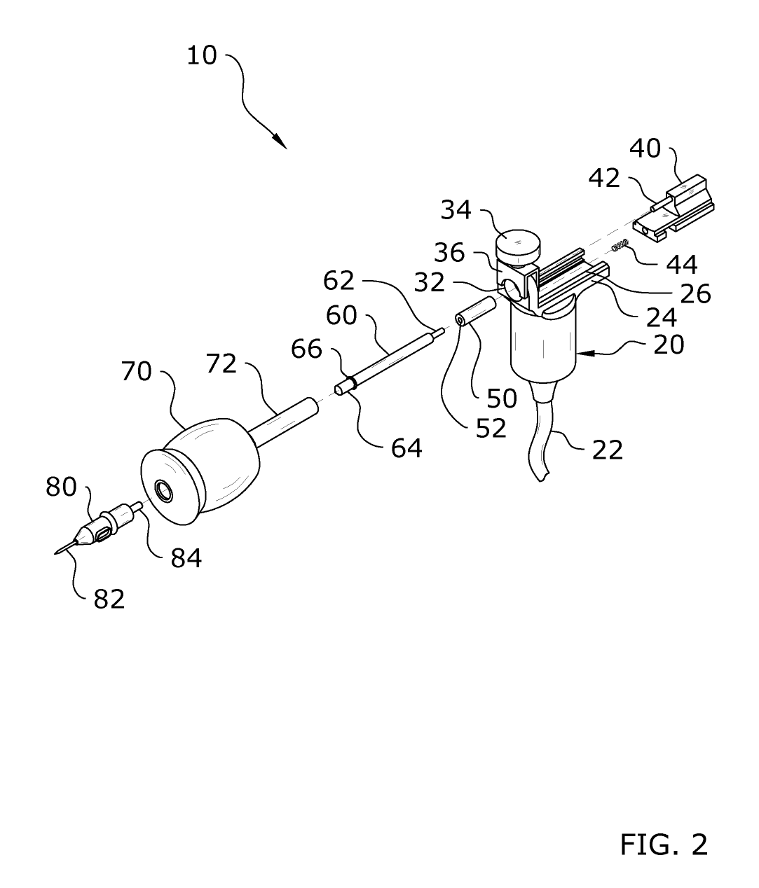

FIGS. 1 through 6 illustrate a preferred embodiment where the drive member 40 includes a drive pin 42 that extends concentrically with respect to the needle 82. The drive pin 42 removably extends into a first opening within the connecting member 50. The first opening within the connecting member 50 preferably extends into the connecting member 50 concentrically to form a passage 52 that removably receives the drive pin 42 as illustrated in FIG. 6. The passage 52 preferably extends completely through the connecting member 50 along a concentric axis of the connecting member 50 from the first opening to the second opening at the opposite end of the connecting member 50. The drive pin 42 is preferably frictionally received within the passage 52 of the connecting member 50 that allows the drive pin 42 to move with respect to the connecting member 50 during the reciprocating motion of the drive member 40. The drive pin 42 preferably extends outwardly from an extended portion of the drive member 40 as best illustrated in FIGS. 2 and 6 of the drawings.

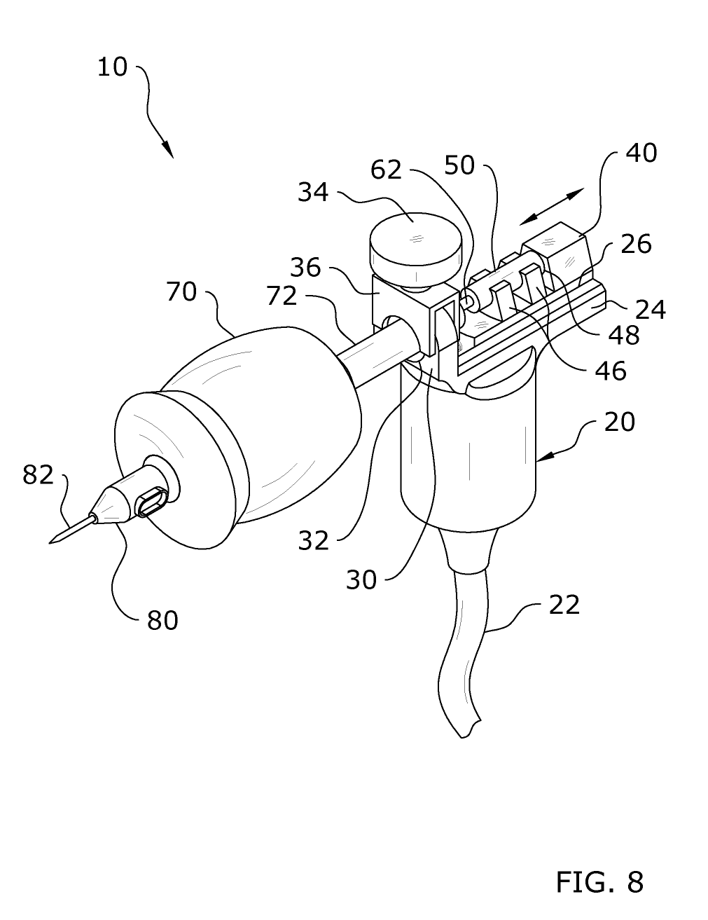

FIGS. 8 through 12 illustrate an alternative embodiment for the drive member 40 that includes at least one receiver bracket 46 that removably receives the connecting member 50. It is preferable that two or more C-shaped brackets extend from the drive member 40 to catchably receive the connecting member 50 in a frictional manner that allows movement of the connecting member 50 within the receiver brackets 46. The extended portion of the drive member 40 forms a stopping wall that prevents movement of the connecting member 50 past the extended portion during the striking movement of the drive member 40. The extended portion preferably includes a recessed portion 48 that is aligned with the connecting member 50 which forms an air cushion during the reciprocating motion of the drive member 40 with the connecting member 50.

FIG. 7a illustrates another alternative embodiment where the drive pin 42 is directly connected to the plunger 60 without the usage of a connecting member 50. FIG. 7b illustrates another alternative embodiment wherein the drive pin 42 extends completely through the connecting member 50 to movably extend through a longitudinal opening within the plunger 60 with the connecting member 50 positioned between the drive member 40 and the plunger 60.

### D. Connecting Member

The connecting member 50 is positioned and connected between the tattoo needle 82 and the drive member 40 to transfer the back-and-forth linear motion from the drive member 40 to the tattoo needle 82. The purpose of the connecting member 50 is to limit the force applied to the tattoo needle 82 by the drive member 40 and to be adjustable by having different types of materials and thicknesses of materials used. While the connecting member 50 is illustrated as being positioned between the plunger 60 and the drive member 40, the connecting member 50 may be positioned between the plunger 60 and the needle engaging member 84.

The connecting member 50 is preferably concentric with respect to the tattoo needle 82. The connecting member 50 is also preferably concentric with respect to the plunger 60 and the drive pin 42 to efficiently transfer the reciprocating force of the drive member 40 in a linear and smooth manner.

The connecting member 50 is preferably comprised of an elongated tube having a passage 52 having a first opening and a second opening as discussed previously and illustrated in FIGS. 2 and 6 of the drawings. However, the connecting member 50 may be comprised of a solid non-tubular structure (e.g. a solid shaft of rubber) or a portion of the connecting member 50 may be tubular with another portion solid and not tubular.

The connecting member 50 is preferably removable from the tattoo needle 82 and the drive member 40. In particular, the connecting member 50 may be replaced at any time with the same type of connecting member 50 or a different type of connecting member 50 having a different resiliency (e.g. softer, firmer, harder, etc.). The connecting member 50 has a first opening that removably receives the drive pin 42 and an opposite second opening that receives the first end 62 of the plunger 60 in a removable manner. The desired firmness and resiliency for the connecting member 50 can be changed by changing the length of the connecting member 50 (e.g. making the connecting member 50 longer to soften the impact), the material type (e.g. using a softer material to soften the impact), the thickness of material (e.g. increasing the thickness of the wall of the tube material to harden the impact) and the like.

The connecting member 50 is constructed of a resilient material. The connecting member 50 is further preferably comprised of an elastic and compressible material that is resilient. Examples of suitable resilient materials include plastic, rubber (e.g. natural rubber or synthetic rubber), elastomers, and other suitable materials having different resiliencies. The connecting member 50 is preferably not a rigid material (e.g. metal or hard plastic). However, there may be situations where the connecting member 50 may be comprised of a rigid and non-resilient material (e.g. metal or hard plastic) where the tattoo artist desires to limit the dampening effect of the connecting member 50 between the drive member 40 and the tattoo needle 82.

The connecting member 50 is preferably color coded to indicate the type of firmness and resilience characteristic for a particular connecting member 50 (e.g. transparent, non-transparent, black, white, gray, red, blue, pink, tan, etc.). For example, a tan color can indicate a solid hit to the tattoo needle 82, a black color can indicate a firm hit to the tattoo needle 82, a transparent color can indicate a soft hit to the tattoo needle 82 and a white color can indicate a very soft hit to the tattoo needle 82 wherein the structure and/or material types for the corresponding connecting members 50 have the corresponding resiliency characteristics. Patterns and other indicia may also be used on the connecting member 50 to indicate the type of firmness and resilience characteristics for a particular connecting member 50 (e.g. numerals and/or letters may be used such as 10 for a solid hit, 7 for a firm hit, 4 for a soft hit and 1 for a very soft hit).

### E. Plunger

The plunger 60 is preferably comprised of a rigid non-resilient material (e.g. metal, hard plastic) and has an elongated structure as best illustrated in FIG. 2 of the drawings. The plunger 60 is positioned between the connecting member 50 and the tattoo needle 82. The plunger 60 is at least partially positioned within the support tube 72 and is concentrically aligned with the tattoo needle 82 to drive the tattoo needle 82 into the skin of the user.

The plunger 60 includes a first end 62 that is removably connected to the connecting member 50 opposite of the drive member 40. The plunger 60 is preferably not in direct physical contact with the drive member 40 or the drive pin 42 to allow the resiliency of the connecting member 50 to provide a slight absorption of the driving force of the drive member 40 during the striking movement. The first end 62 of the plunger 60 is preferably narrower and has a width that fits within the second opening of the connecting member 50 as illustrated in FIG. 6 of the drawings. The first end 62 and the main body of the plunger 60 form a shoulder that the connecting member 50 abuts during operation of the present invention.

The plunger 60 preferably includes a groove that surrounds the perimeter of the plunger 60 near the second end 64 that receives an O-ring 66 which supports the plunger 60 in a concentric manner within the tube. The distal end of the second end 64 preferably has a tapered structure that corresponds to a tapered recessed portion 48 for the end of the needle engaging member 84 as illustrated in FIG. 6 of the drawings.

### F. Needle Cartridge

The needle cartridge 80 is preferably removably attached to a distal end of the support tube 72. The needle cartridge 80 is further preferably comprised of a disposable needle cartridge 80 for sanitation purposes which can be disposed of after usage and replaced with a new needle cartridge 80. While the needle cartridge 80 is preferably removably connected to the support tube 72 or drive unit 20, the needle cartridge 80 may be integrally attached to the support tube 72 or drive unit 20 in a non-removable manner. U.S. Pat. No. 6,505,530 to Adler et al. and U.S. Pat. No. 8,029,527 to Lisec illustrate examples of suitable needle cartridges 80 for the present invention and are hereby incorporated by reference herein.

The needle cartridge 80 includes a tattoo needle 82 that is preferably concentrically aligned with respect to the support tube 72. The tattoo needle 82 reciprocates back-and-forth from a distal end of the needle cartridge 80 to penetrate the skin to apply the ink to the skin. The needle cartridge 80 further includes a needle engaging member 84 that is concentrically aligned with and connected in a non-movable manner to the tattoo needle 82 that extends outwardly from the inner end of the needle cartridge 80. A spring within the needle cartridge 80 applies a retracting force to the tattoo needle 82 and the needle engaging member 84 to retract the tattoo needle 82 from the skin when the drive member 40 is in the retracted position.

### G. Operation of Preferred Embodiment

In use, the tattoo artist determines the desired firmness for the needle strike into the skin of the customer for a particular tattoo procedure and selects the appropriate connecting member 50. For example, for a soft hit the tattoo artist selects a connecting member 50 with a soft hit characteristic, for a hard hit the tattoo artist selects a connecting member 50 with a hard hit characteristic and so forth. The tattoo artist then attaches the connecting member 50 between the drive member 40 and the tattoo needle 82. As illustrated in FIGS. 1, 3, 4 and 6 of the drawings, the selected connecting member 50 is attached to the drive pin 42 of the drive member 40 at one end and to the first end 62 of the plunger 60 at the opposite end. The tattoo artist secures the support tube 72 and then proceeds with the tattoo procedure on the customer with the tattoo needle 82 having the corresponding firmness of needle strike based upon the selected connecting member 50. If the tattoo artist desires to change the firmness of the needle strike, the tattoo artist simply removes the first selected connecting member 50 and attaches a second selected connecting member 50 with the different firmness of needle strike. Various levels of firmness for the needle strike may be achieved utilizing various connecting members 50.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A tattoo machine system, comprising:
a drive unit having a frame and a motor;
a drive member movably connected to said drive unit, wherein said motor is mechanically connected to said drive member and wherein said drive member is adapted to reciprocate in a back-and-forth linear motion;
a tattoo needle; and
a connecting member connected between said tattoo needle and said drive member to transfer said back-and-forth linear motion from said drive member to said tattoo needle, wherein said connecting member is comprised of a resilient material and wherein said connecting member is removable from said tattoo needle and said drive member.

2. The tattoo machine system of claim 1, wherein said motor is a rotary motor.

3. The tattoo machine system of claim 2, wherein said rotary motor is an electric motor.

4. The tattoo machine system of claim 1, wherein said connecting member is color coded according to a resilience characteristic for said connecting member.

5. The tattoo machine system of claim 1, wherein said connecting member is comprised of a tube having a passage.

6. The tattoo machine system of claim 1, wherein said connecting member is comprised of an elongated structure.

7. The tattoo machine system of claim 1, wherein said connecting member is concentrically aligned with said tattoo needle.

8. The tattoo machine system of claim 1, wherein said drive member is slidably positioned in a channel of said drive unit.

9. The tattoo machine system of claim 1, wherein said drive member includes a drive pin that extends concentrically with respect to said needle, wherein said connecting member includes a passage that receives said drive pin.

10. The tattoo machine system of claim 1, wherein said drive member includes at least one receiver bracket that removably receives said connecting member.

11. A tattoo machine system, comprising:
a drive unit having a frame and a motor;
a support tube attached to said drive unit;
a needle cartridge removably attached to a distal end of said support tube, wherein a tattoo needle of said needle cartridge is concentrically aligned with respect to said support tube;
a drive member movably connected to said drive unit, wherein said motor is mechanically connected to said drive member and wherein said drive member is adapted to reciprocate in a back-and-forth linear motion along a first axis that is parallel to a longitudinal axis of said tattoo needle; and
a connecting member connected between said tattoo needle and said drive member to transfer said back-and-forth linear motion from said drive member to said tattoo needle, wherein said connecting member is comprised of a resilient material, wherein said connecting member is removable from said tattoo needle and said drive member and wherein said connecting member is concentric with respect to said tattoo needle.

12. The tattoo machine system of claim 11, wherein said motor is a rotary motor.

13. The tattoo machine system of claim 12, wherein said rotary motor is an electric motor or pneumatic motor.

14. The tattoo machine system of claim 11, wherein said connecting member is color coded according to a resilience characteristic for said connecting member.

15. The tattoo machine system of claim 11, wherein said connecting member is comprised of an elongated tube having a passage.

16. The tattoo machine system of claim 11, wherein said connecting member is comprised of a plastic material or a rubber material.

17. The tattoo machine system of claim 11, wherein said drive member is slidably positioned in a channel of said drive unit.

18. The tattoo machine system of claim 11, wherein said drive member includes a drive pin that extends concentrically with respect to said needle, wherein said connecting member includes a passage that receives said drive pin.

19. The tattoo machine system of claim 11, wherein said drive member includes at least one receiver bracket that removably receives said connecting member.

20. A tattoo machine system, comprising:
a drive unit having a frame and a motor;
a support tube attached to said drive unit;
a needle cartridge removably attached to a distal end of said support tube, wherein a tattoo needle of said needle cartridge is concentrically aligned with respect to said support tube;
a drive member movably connected to said drive unit, wherein said motor is mechanically connected to said drive member and wherein said drive member is adapted to reciprocate in a back-and-forth linear motion along a first axis that is parallel to a longitudinal axis of said tattoo needle;
a connecting member connected between said tattoo needle and said drive member to transfer said back-and-forth linear motion from said drive member to said tattoo needle;
wherein said connecting member is comprised of a resilient material;
wherein said connecting member is removable from said tattoo needle and said drive member;
wherein said connecting member is concentric with respect to said tattoo needle;
wherein said connecting member is comprised of an elongated tube having a passage having a first opening and a second opening;
wherein said connecting member is comprised of a plastic material or a rubber material;
a plunger having an elongated structure, wherein said plunger is positioned between said connecting member and said tattoo needle, wherein said plunger is at least partially positioned within said support tube, and wherein said plunger is concentrically aligned with said tattoo needle.
